(19) United States
(12) Patent Application Publication
(10) Pub. No.: US 2026/0272295 A1
(43) Pub. Date: Sep. 17, 2026

(54) DEVICE FOR MEASURING PROPERTIES OF EYE AND METHOD THEREOF

(71) Applicant: ICARE FINLAND OY, Vantaa (FI)

(72) Inventor: Andrei Plaian, Noventa Padovana, Pd (IT)

(73) Assignee: ICARE FINLAND OY, Vantaa (FI)

(21) Appl. No.: 19/168,045

(22) PCT Filed: Mar. 21, 2024
(86) PCT No.: PCT/FI2024/050134

§ 371 Date: Sep. 23, 2025

(30) Foreign Application Priority Data
Apr. 25, 2023 (FI) 20235457

(51) Int. Cl. A61B 3/16; A61B 3/10

(52) U.S. Cl. CPC A61B 3/16; A61B 3/1005

(57) ABSTRACT

A device for measuring properties of an eye. The device includes: an execution unit including an impact means arranged to apply an impact on a cornea of the eye, wherein the execution unit includes launching means, and solid probe(s), a measuring unit including at least one confocal chromatic displacement sensor, and a controller coupled to the execution unit and the measuring unit, wherein the controller is configured to operate the execution unit to apply the impact on the cornea of the eye using the impact means, and the measuring unit to perform multiple measurements of a displacement of the cornea caused by the impact during a predetermined time period, the launching means is operable to launch the at least one solid probe towards the eye to apply the impact.

### TECHNICAL FIELD

The present disclosure relates to a device for measuring properties of an eye. The present disclosure also relates to a method for measuring properties of an eye.

### BACKGROUND

Tonometers are devices that measure an internal eye pressure (e.g. intra-ocular pressure (IOP)). Devices which measure central cornea thickness (CCT) are referred as pachymeters. Said devices are used for measuring various properties of an eye of subject.

The measurement results can be used for example to make diagnoses or to follow up the evolution in time of certain property or parameter of an eye. As an example the measured eye properties (such as IOP and/or CCT) help in diagnosing and managing various eye conditions, including glaucoma, which can lead to vision loss if left untreated. Notably, there exist various types of the tonometers and various types of pachymeters that work on different principles. A rebound tonometer is one example of a tonometer. The rebound tonometer can be used to determine indication of the intra ocular pressure (IOP) by launching a probe towards eye and measuring a velocity profile of the probe during an impact of the probe with a surface of the eye. The velocity profile can be used to determine the IOP. As an example, low change of speed (dv/dt i.e. acceleration) indicates low IOP. Faster change of speed during the impact on the other hands indicates higher IOP. An example of pachymeter is a pachymeter using confocal microscopy. Other example of pachymeter is a pachymeter using ultrasound to measure properties of eye. Current situation has a problem that tonometer and pachymeter are separate devices. The operation and usability of a group of two separate devices is worse than those of a single device able to perform both functions. Also a single device costs might be lower than multiple different devices.

As a further problem with a device for measuring IOP using rebound tonometer is that elastic characteristics of the eye might be also a function of central corneal thickness (CTT). Therefore, IOP measurement done using prior art way might provide at least partially wrong values.

Additional way to measure IOP is causing the eye surface (cornea in practice) to oscillate and measure a frequency of free oscillations of the cornea caused by the impact. The frequency of free oscillations of the cornea caused by the impact can be used to determine IOP value. Currently there exist no devices, which can be used in easy and convenient way to use the frequency of free oscillations of the cornea caused on by the impact to obtain (or to calculate) IOP values.

### SUMMARY

The present disclosure seeks to provide a device for measuring properties of an eye. One of the measured properties is an intra ocular pressure (IOP) of the eye. According to present disclosure the measurement is done by applying an impact on a cornea of the eye. The impact causes the cornea to start oscillating in its frequency of free oscillations of the cornea caused by the impact (also known as a natural frequency). It has been found out that IOP is function of said frequency of free oscillations of the cornea caused by the impact. In the present disclosure a measurement unit is used to measure displacement of the cornea caused by the impact. The measurement is done over a predetermined period of time. This way, the frequency of free oscillations of the cornea caused by the impact, can be measured using the measurement unit. Practically, the frequency of free oscillations of the cornea cause by the impact is equal or very close to the resonance frequency (or natural frequency) of cornea. Indeed, there might be some small difference between the frequency of oscillation of the cornea caused by the impact and the resonance frequency of the cornea, during the impact or suddenly after, when the amplitude of oscillation is large. Instead, for smaller amplitudes, the cornea oscillates on its resonance frequency.

In one aspect, an embodiment of the present disclosure provides a device for measuring properties of an eye, the device comprising:
an execution unit comprising an impact means arranged to apply an impact on a cornea of the eye,
   wherein the execution unit comprises
   a launching means, and
   at least one solid probe,
a measuring unit comprising at least one confocal chromatic displacement sensor, and
a controller coupled to the execution unit and the measuring unit,
   wherein the controller is configured to operate:
the execution unit to apply the impact on the cornea of the eye using the impact means, and
the measuring unit to perform multiple measurements of a displacement of the cornea caused by the impact during a predetermined time period,
   wherein the launching means is operable to launch the at least one solid probe towards the eye to apply the impact.

In another aspect, an embodiment of the present disclosure provides a method for measuring properties of an eye, the method comprising:
creating an impact on a cornea of the eye using an execution unit comprised in a device, and
performing, using a measuring unit, multiple measurements of a displacement of the cornea caused by the impact during a predetermined time period, wherein the measuring unit comprising at least one confocal chromatic displacement sensor,
   wherein the impact on the cornea is created by launching at least one solid probe towards the eye, the at least one solid probe being comprised in the execution unit.

Embodiments of the present disclosure substantially eliminate or at least partially address the aforementioned problems in the prior art, and enable an improved, simple, compact, accurate, reliable and cost-efficient device for measuring properties of eye. Beneficially, the device impacts only the cornea of the eye thereby inducing the desired oscillations only in the cornea. Moreover, the device employs the at least one confocal chromatic displacement sensor in order to enable the measurement of the properties of the eye with a better precision.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, similar elements have been indicated by identical numbers.

Embodiments of the present disclosure will now be described, by way of example only, with reference to the following diagrams wherein:

FIG. 1A is a block diagram illustrating a device for measuring properties of an eye, in accordance with an embodiment of the present disclosure;

FIG. 1B is a block diagram illustrating an execution unit of the device, in accordance with an embodiment of the present disclosure;

FIG. 2 is a block diagram illustrating an execution unit of a device, in accordance with another embodiment of the present disclosure;

FIG. 3 is a block diagram illustrating a device, in accordance with the other embodiment of the present disclosure;

FIG. 4 is a block diagram illustrating a device, in accordance with the other embodiment of the present disclosure;

FIG. 5 is a graphical representation depicting a frequency with respect to the internal pressure value of an eye, in accordance with the first preferred embodiment of the present disclosure;

FIG. 6 is a flowchart depicting steps of a method for measuring properties of an eye, in accordance with an embodiment of the present disclosure; and

FIG. 7 is an example according to one embodiment of the present disclosure.

In the accompanying drawings, an underlined number is employed to represent an item over which the underlined number is positioned or an item to which the underlined number is adjacent.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a device for measuring properties of an eye, the device comprising:
an execution unit comprising an impact means arranged to apply an impact on a cornea of the eye,
   wherein the execution unit comprises
a launching means, and
at least one solid probe,
a measuring unit comprising at least one confocal chromatic displacement sensor, and
a controller coupled to the execution unit and the measuring unit,
   wherein the controller is configured to operate
the execution unit to apply the impact on the cornea of the eye using the impact means, and
the measuring unit to perform multiple measurements of a displacement of the cornea caused by the impact during a predetermined time period,
   wherein the launching means is operable to launch the at least one solid probe towards the eye to apply the impact.

In another aspect, an embodiment of the present disclosure provides a method for measuring properties of an eye, the method comprising:
creating an impact on a cornea of the eye using an execution unit comprised in a device,
performing, using a measuring unit, multiple measurements of a displacement of the cornea caused by the impact during a predetermined time period, wherein the measuring unit comprises at least one confocal chromatic displacement sensor,
   wherein the impact on the cornea is created by launching at least one solid probe towards the eye, the at least one solid probe being comprised in the execution unit.

The device can be used to measure properties of the eye. One property to be measured is displacements of the cornea as function of time. The displacement as function of time provides information which can be further used to determine further properties. Present device provides impact with a launching means to a cornea of the eye. As an example, electromagnetic force is used to eject a solid probe towards eye. The probe bounces back after the impact. Resulting displacement of the cornea is measured with confocal chromatic sensor during and after the impact. As an example, this displacement as function of time can be used to find frequency of free oscillations of the cornea. The frequency on the other hand correlates with intraocular pressure (IOP) of the eye.

One of the benefits of measuring, together with one device, both IOP and CCT, has the advantage that the CCT value may be used to apply a correction during the calculation of the IOP, therefore leading to a more precise value of IOP. Another advantage of having both the measurements made by the same device is that one single device is easier to use and cost less than two separate devices that measure separately the IOP and the CCT.

Indeed, the present disclosure provides a device that measures properties of an eye. The device comprises an excitation unit which comprises impact means. The applied impact causes the cornea to start vibrating (or oscillating). The device then measures the vibrational behavior of the cornea with a measuring unit. The measuring unit uses as measurement means, a confocal chromatic displacement sensor (CCS). In practical terms CCS performs a series of measurements of the displacement of the cornea caused by the impact over a period of time.

This series of displacements of the cornea over a predetermined period of time can be used to find the frequency of free oscillations of the cornea caused by the impact. Indeed, the measured displacements can be thus used to find the frequency of free oscillations of the cornea caused by the impact and further used to calculate the eye pressure based on the frequency of the free oscillations of the cornea caused by the impact.

As a further advance of using CCS as measurement means is that the device measures not only the pressure (tonometer function), but also the cornea thickness (pachymeter function). The measurement of the cornea thickness is possible since the CCS is able to simultaneously measure the position of the external surface of the cornea and the internal surface of the cornea. The device can be thus considered to be a multi-functional device that measures two properties of an eye at once (CCT and IOP).

As an additional benefit, the measured CCT value may be used to apply a correction on the calculation of the IOP in order to improve the precision of calculation of the IOP value. Additionally, the device employs the at least one confocal chromatic displacement sensor that provides precise measurement of the properties of the eye.

Pursuant to the embodiments of the present disclosure, the term “device” as used herein refers to an instrument that is used for measuring various properties of an eye. In this regard, the properties refer to physiological parameters associated with the eye. Optionally, properties such as intra ocular pressure (or in other words, internal pressure value of the eye) (IOP) and central corneal thickness (CCT) can be measured with the device. As a further advantage of the device (having multiple functionalities), we can use the device as contactless device for measuring the central corneal thickness values.

The term “execution (or excitation) unit” as used herein refers to an arrangement that is used for applying impact on the cornea of the eye when in use. The term “impact means” as used herein refers to a means that is used for impacting a surface. The impact means is arranged inside the execution unit. The impact means refers to means which can be used to provide the impact on the surface. As an example, the impact means can comprise an electric coil and a probe having magnetic elongated body. In this example the electric coil is used to accelerate a probe towards an eye. Thus the probe is launched/ejected from the execution unit towards a eye to collide with the eye. In an example, the impact means is thus used for impacting the surface of the eye. It will be appreciated that the impact means is arranged to create the impact only on the cornea of the eye in order to cause local oscillations of the cornea.

The term “measuring unit” as used herein refers to a component that is used for measuring oscillations of the cornea of the eye. An example of the measuring unit is a measurement unit which comprises at least one confocal chromatic displacement sensor (CCS). The confocal chromatic displacement sensor is arranged on the device in such a way that it can be used to measure oscillations caused by the impact means. A further advantage of using confocal chromatic displacement sensor is that it has fast response time and accuracy. Displacement value of cornea caused by applied impact is in sub millimeter range. Confocal chromatic sensors are able to measure displacements even with tens of nanometers resolution, therefore are able to measure the cornea oscillations caused even by a very weak impact. Fast measuring frequency is needed to be able to make sufficient amount of measurements in the predefined period of time to find out a frequency of oscillations (i.e. the measurement frequency should be at least double (or higher, for example 3, 5, 10 etc. times) with respect to the frequency of oscillation of the cornea as per Nyquist sampling theorem) CCS might have measurement frequencies of thousands of measurements per second, which is high compared to a typical frequency of free oscillations of the cornea that is in the range of hundreds of hertz. Therefore, CCS can be used to measure vibrational behavior of the cornea after impact directly.

The term “confocal chromatic displacement sensor” as used herein refers to an instrument that works by focusing a beam of light with a wide band of wavelengths, usually white light, onto a target surface (such as the eye) using a high dispersion objective lens. In this regard, the measuring unit includes the at least one confocal chromatic displacement sensor. It will be appreciated that the at least one confocal chromatic displacement sensor is placed at an appropriate position, for identifying the oscillations of the cornea accurately. Optionally, the beam of light is preferably oriented normally to the cornea. Advantageously, the at least one confocal chromatic displacement sensors possess the ability to tolerate a large inclination of an axis of the beam with respect to the normal direction of the measured surface (even tens of degrees). As a result, even if the device is hand-held and not properly centered or oriented, the measurement may not be affected. It will be appreciated that the at least one confocal chromatic displacement sensor may be used for resolutions of measurement of the displacement of tens of nanometers at a measuring frequency of tens of kilohertz (KHz). Moreover, the at least one confocal chromatic displacement sensors allow precise measurement of the position of the at least one point of the cornea. Beneficially, the at least one confocal chromatic displacement sensor may be customized with the goal to only identify the frequency of oscillations of the cornea, in order to make it cost-efficient. Optionally, the at least one confocal chromatic displacement sensor uses infrared light (IR) as a measuring beam. Beneficially, the IR light is not seen by the eye of the patient. In such a case, the temptation of the subject to blink the eye is reduced during the initial phase when setting a right relative position between the device and the eye. Additionally, the infrared light is less likely to cause damage to the eyes being measured. Beneficially, the at least one confocal chromatic displacement sensor is able to measure the thickness of the cornea, as it possesses the ability to simultaneously measure the position of both an external surface and an internal surface of the cornea. The cornea thickness may be calculated as the difference between the position of the external surface of the cornea and the position of its internal surface, multiplied by the refraction index of the cornea. Furthermore, the at least one confocal chromatic displacement sensor has a high resolution, which allows it to detect even small values of amplitude of oscillation of the cornea. Typically, CCS is arranged to perform measurements on a single point of the cornea. Additionally, the at least one confocal chromatic displacement sensors do not require contact with the cornea, which can reduce the risk of infection and discomfort for the subject. Furthermore, the at least one confocal chromatic displacement sensors enable fast measurement of the properties of the eye. Furthermore, the at least one confocal chromatic displacement sensors makes the device user-friendly and does not require extensive training to operate, which can make it more accessible to healthcare providers in various settings.

The term “controller” refers to a computational device that is operable for controlling the overall operation of the device. The controller, in operation, performs tasks such as, but not limited to, using the execution unit, using the measuring unit and responding to and processing information. In an example, the controller may contain an embedded microcontroller, a microprocessor, or the like. In this regard, the controller is coupled with the measuring unit and the execution unit. The controller may be implemented as an internal component of the device, an external component of the device, or a combination thereof.

The controller is configured to operate the execution unit by energizing it. In this regard, the execution unit is energized in order to apply the impact on the cornea of the eye. Notably, the eye is considered to be a thin-walled elastic vessel filled with a pressured fluid. As a consequence, the eye has a mechanical vibrational behavior and a resonance frequency. In present disclosure we measure the frequency of free oscitations of the cornea caused by the impact. This is a good representation of the resonance frequency and can be thus used for determining IOP. Moreover, when the cornea experiences the impact, it can deform and then rapidly return to its original shape, creating oscillations in its position. The oscillation is the evolution in time of the position (displacement) of the cornea.

Moreover, the controller is configured to operate the measuring unit. The measuring unit is used for performing multiple measurements of the displacement of the cornea that are caused by the impact during a predetermined time period. Herein, the multiple measurements refer to a series of measurements of the displacement of the cornea over a predetermined period of time. In this regard, the multiple measurements provide information of displacement as function of time. This can be used to determine oscillation frequency. In addition the measurement will provide information on amplitude of the oscitations. Optionally, the multiple measurements enable tracking of a progression of the displacement of the cornea over time and get a better understanding of the impact's effects on the cornea. Herein, the displacement refers to a change in a position of the cornea caused by the oscillations thereof. The frequency of the multiple measurements is high enough to identify the oscillations of the cornea. As an example, multiple measurements (plurality of measurements) with the displacement sensor could be for example 800 measurements per second as the frequency of free oscillations is typically less than 400 Hz. Typical, expected, frequency of corneal oscillations is in range of 240 Hz to 400 Hz i.e. multiple measurements refer to making in range of 800 measurements per second. In order to have more precise information of the oscillations a sampling rate of taking the multiple measurements could be 1000 measurements per second or higher such as 2000 measurements of the displacement of the cornea. The predetermined time can be for example a time needed for the cornea oscillations to stop (or reduce to certain amplitude level).

Optionally, the device further comprises a calculating unit coupled to the controller and operable to
calculate a frequency of free oscillations of the cornea caused by the impact using the multiple measurements of the displacement of the cornea;
calculate an internal pressure value of the eye as a function of the frequency of the free oscillations of the cornea caused by the impact.

The term “calculating unit” as used herein refers to an electronic hardware or a software algorithm that is used for performing mathematical operations to convert raw data into a meaningful measurement. In this regard, the calculating unit is associated with the controller in order to allow for greater flexibility and customization in the device based on a design thereof. Optionally, the calculating unit is integrated into the controller. Moreover, the calculating unit is operable to calculate a frequency of the free oscillations of the cornea induced by the impact based on the performed series of measurements. The calculating unit is further operable to use the calculated frequency of free oscillations of the cornea caused by the impact to determine the internal pressure value of the eye using the value of said frequency of free oscillations of the cornea caused by the impact. The cornea performs the free oscillations with a decreasing amplitude until it stops. The frequency of free oscillations of the cornea caused by the impact is correlated to the internal pressure of the eye. Alternatively, the calculating unit can be provided remote from the device. It can be for example implemented in a smart phone which is communicatively connected to the device. Furthermore, the calculating unit can be implemented in a server system remote from the device. In said scenario measurements of the measurement unit are provided to server (or to the smart phone depending on the case) and the calculation of IOP and other possible parameters is done in said server. In essence, intelligence can be distributed between the device and external calculation unit. One way to calculate from the free osciallations the internal pressure is to form physiological model of the eye and use the oscillation frequency, impact (of the probe) values etc as input and calculate the pressure. Other way to calculate IOP is to use look up table which provides oscillation frequencies and related IOP values.

The term “internal pressure of the eye” (namely an intraocular pressure (IOP)) as used herein refers to a fluid pressure of the eye. Optionally, the calculating unit may apply different mathematical models, such as regression analysis or curve fitting, to the raw data to obtain the most accurate and reliable measurements of the internal pressure value of the eye. Optionally, the calculating unit may perform other functions, such as error correction, data storage, and data analysis. Notably, the internal pressure value of the eye is used to diagnose overall eye health and function. Optionally, the eye behaves like a mass-spring system, where the IOP describes the spring constant. Optionally, when the cornea is displaced from its natural position by an external force, it vibrates as a damped harmonic oscillator with a frequency relative to the IOP. The IOP modulates the stiffness of the eye as well as the cornea so that the oscillation frequency increases with increasing IOP. It will be appreciated that the measurement of the internal pressure value enables a diagnosis and treatment of ocular hypertension before the development of eye-related conditions.

Optionally, the measuring unit is further configured to measure a thickness value of the cornea at least at one point on the cornea. In this regard, the measuring unit is operable to measure the thickness of the cornea at one or more points of the cornea. Beneficially, the thickness is measured using the measuring unit, as it possesses the ability to simultaneously measure the position of both an external surface and an internal surface of the cornea. Moreover, the thickness of the cornea characterizes the stiffness of the cornea and therefore it affects the frequency of free oscillations of the cornea caused by the impact together with the IOP. Technical effect of including the thickness value when determining IOP from frequency of free oscillations of the cornea caused on the impact is that it makes the determined value more accurate.

Optionally, the calculating unit is further operable to apply a correction to the calculated internal pressure value based on the measured cornea thickness value. In this regard, the thickness of the cornea characterize the stiffness of the cornea, which is a disturbing factor when measuring the internal pressure of the eye. Moreover, when the thickness of the cornea is measured, its value may be used to apply a correction when calculating the internal pressure value of the eye. For example, the thickness is correlated to a frequency of free oscillations of the cornea caused by the impact which is then used for calculating the internal pressure value of the eye. This allows to calculate the internal pressure of the eye with improved precision. As an example, a table I (below) values can be used as a basis for the correction calculation if a central corneal thickness is measured. For example if the thickness is 485 micrometers then the IOP value is corrected by adding 3 mmHg. The table values should be modified if the measurement point is not central. The table can be used as look up table or, for example, a correction value as function of the corneal thickness can be derived from the values. Technical effect of this is that we can get more accurate measurements of intra ocular pressure.

TABLE I
Example of IOP value corrections as
function of central corneal thickness

| Cornea Thickness (micrometers) | Correction Values (mm Hg) |
|---|---|
| 465 | 4 |
| 485 | 3 |
| 505 | 2 |
| 525 | 1 |
| 545 | 0 |
| 565 | −1 |
| 585 | −2 |
| 605 | −3 |
| 625 | −4 |

Moreover, the execution unit further comprises
a launching means, and
at least one solid probe,
   wherein the launching means is operable to launch the at least one solid probe towards the eye to create (or, in other words, to apply) the impact.

The term “launching means” as used herein refers to a mechanical element that is used to launch (or in other words project, throw, eject) the at least one solid probe from the execution unit with a certain velocity and in a certain direction. In this regard, the launching means enables an efficient and accurate movement of the at least one solid probe, when in use. Optionally, the launching means partially surrounds the at least one solid probe. The at least one solid probe can comprise for example an elongated magnetic body and a tip part at end of the elongated magnetic body. Herein, the launching means is arranged as a solenoid containing a set of loops through which the at least one solid probe can move. The launching means moves the at least one solid probe when electric current is fed through the launching means (due to electromagnetic force applied by the magnetic field to the elongated magnetic body of the probe). The launching means optionally ejects the magnetic elongated probe and projects the at least one solid probe towards the eye with a predetermined velocity. As the probe is ejected towards the eye the tip of the probe eventually hits the eye causing cornea of the eye to oscillate. The oscillation frequency will be the free oscillations frequency of the cornea caused by the impact. The probe will rebound back from the eye after the impact. The launching means can be further configured to pull back the probe inside of the execution unit or launching means preparing it for a next measurement. It will be appreciated that the launching means and the at least one solid probe work in conjunction with each other to create an accurate impact on the cornea of the eye meaning that the velocity of the probe is the desired one and the impact is applied on the right point on the cornea. The described example of the at least one solid probe and the electromagnetic launching mechanism are not intended as the unique possible types of the at least one solid probe and the launching mechanisms. It is possible to use another type of the at least one solid probe and another type of the launching mechanism based on another operating principle.

As an example, the impact on the cornea with a solid probe generates several oscillations of the cornea during the period of time from the impact to the moment that the oscillation stops. By means of the CCS it is possible to acquire a series of displacement measurements during a period of time that may comprise a number of said several complete free oscillations induced/applied by the impact, or even all of them. Therefore, this technique is less subject to measurement errors when compared to a known type rebound tonometer, since it allows to calculate a frequency of the free oscillations of the cornea induced by the impact as a mean value calculated in a longer time, comprising a number of entire periods of cornea oscillation. Instead, the known rebound tonometer performs its measurement only during the impact, in a much shorter time of about half period of oscillation of the cornea. The frequency of the free oscillations of the cornea induced by the impact and measured with the CCS is related to the IOP and allows the calculation of the IOP. Therefore, the present disclosure provides a more precise solution.

It will be appreciated that the device provides an accurate determination of a stimulus site within the eye. In this regard, as a result of launching the at least one solid probe towards the eye to apply the impact, the cornea is stimulated (or displaced) and the stimulus site is formed. Thus, the determination of the stimulus site can be done accurately. Further, the measurement of displacement at the stimulation site is done with higher accuracy and reliability. Moreover, the at least one solid probe hits the cornea, and the measurement is performed at the stimulation site. The impact application, facilitated by the launching means, allows for consistent and reproducible measurements of the cornea's displacement during the predetermined time period using the confocal chromatic displacement sensor. Such a configuration enables the calculation of essential parameters such as the frequency of free oscillations of the cornea and subsequent determination of the eye's internal pressure value. Since the probe is solid and has determined dimensions (such as area of the tip part which will collied with a surface of the eye) resulting impact (and force and pressure of the impact) from the collision can be controlled accurately. If value of impact (Newton sec) is required to be changed a speed of the probe can be adjusted accordingly. Also probe type can be changed to have a probe which have different weights or geometrical dimensions. For example if speed of probe and weight of probe upon impact is same but tip area is reduced there is larger pressure impacting the eye in smaller area. This might result to oscillation pattern which is easier to measure than with larger tip. Optionally, the execution unit further comprises
a launching means, and
at least one drop of liquid,
   and wherein the launching means is operable to launch the at least one drop of liquid towards the eye to create (or in other words to apply) the impact.

Herein, the launching means refers to another mechanical element that is used to release or launch one or more drops of the liquid towards the eye to create the impact on the cornea of the eye. Example of launching means to launch one or more drips of liquid can include a pressured liquid container having an electrically controlled valve and a nozzle arranged towards eye (when in use). As the valve is controlled a droplet of liquid is ejected from the nozzle. Alternatively for example a piston type of arrangement can be used to eject liquid. Optionally, the at least one drop of liquid is selected from at least one of: water, a salted water or a physiological solution. Optionally, the at least one drop of liquid is water. Optionally, the drop of water could be used as a non-irritating liquid that can be launched towards the eye without causing harm or discomfort thereto. Optionally, the at least one drop of liquid is the salted water. Typically, the salted water is a sterile solution of water and salt that is used for various purposes such as an eye irrigation. The eye irrigation is a process of flushing out the eye with a stream of fluid in order to remove foreign objects, irritants, or chemicals that may have entered the eye. Optionally, the at least one drop of liquid is a Physiological Solution, which is a solution of a salt (an approximately 0.9 percent solution of sodium chloride) that may contain other salts and that is essentially isotonic with tissue fluids or blood. In this regard, when in operation, the controller is configured to operate the execution unit in such a manner that the launching means associated therewith launches the at least one drop of liquid towards the eye in order to create an impact and oscillations of the cornea. The frequency of the free oscillations of the cornea after the impact is indicative of the value of the IOP. Notably, a higher IOP is indicative of an increased stiffness of the eyeball, and therefore an increased frequency of the free oscillations of the cornea after the impact is observed in the measurements. And vice versa i.e. lower IOP is indicative of decreased stiffness of the eyeball, and therefore a decreased frequency of the free oscillations of the cornea after the impact is observed.

As discussed according to one embodiment the device launches at least a drop of liquid towards the cornea to generate an impact. The impact of liquid on the surface of eye creates oscillations. The vibrational behavior of the cornea during a time comprising several free oscillations of the cornea induced by the impact can be then measured. This type of measurement allows the calculation of a frequency of the free oscillations of the cornea induced by the impact, which is related to the IOP. A benefit of using liquid drops as impact means is that this can eliminate the possibility to transmit disease. When using liquid one can store sterilized liquid in a container of the device and administer that from the device without risk of contamination.

Optionally, the at least one solid probe is designed to contain the at least one drop of liquid, and wherein the launching means is operable to launch the at least one solid probe towards the eye to deliver the at least one drop of liquid at the cornea of the eye to create the impact. Furthermore, the mechanism to launch droplets might comprise an open container filled with the right quantity of liquid. The container is pushed towards the cornea with a desired velocity and then stopped before touching the cornea, causing the exit of the liquid drop from the container. The liquid drop continues its movement towards the cornea by inertia and finally apply the impact on the cornea One problem when ejecting a small amount of liquid is surface tension of the liquid. For this regard one mean to provide liquid towards eye is to use a piston that expels the drop of liquid through an orifice. This type of arrangement have been found out to provide constant size and speed liquid droplets.

Indifferently of the technique used to apply the impact on the cornea (impact with solid probe or liquid drop), the presence of the CCS sensor used to measure the vibrational behavior of the cornea after the impact allows also to measure the central cornea thickness. This allows to the device not only to perform a tonometer function (measuring of the IOP) but also to perform a pachymeter function (measuring the CCT).

Optionally, the device further comprises
at least one sensor element operable to measure a distance between the cornea of the eye and the device, and
a feedback element, coupled to the at least one sensor element, operable to provide a feedback based on the measured distance,
   wherein the feedback is selected from at least one of: a visual feedback, an acoustic feedback.

Herein, the sensor element refers to a device that is able to measure the position of the cornea and convert the position information into electrical or digital signals that can be measured or processed. Optionally, the at least one sensor element is an internal component of the device or an external component of the device. Optionally, the at least one sensor element can be used alone or in combination with other sensors to provide more complex and detailed measurements of the environment or system being monitored. The term “feedback element” as used herein refers to means that use the output signal provided by the at least one sensor element to generate a feedback signal. The feedback element can be used to provide information useful for the user to correctly position the device relative to the eye and to establish when to perform measurements.

It will be appreciated that the at least one sensor works in conjunction with feedback element to help the human operator to correctly position the device relative to the eye, when the device is a hand-held unit. Moreover, the feedback is provided as a visual feedback. For example, the measured distance is displayed on a screen of a device. Optionally, the visual feedback is represented in the form of a textual information or a graphical information, or a LED light that goes on/of or changes color. Furthermore, the feedback is provided as an acoustic feedback. For example, the feedback is provided to an operator of the device as an audio feedback via a speaker associated with a device. Advantageously, the visual feedback or the acoustic feedback is used in order to assist the correct positioning of the device performed by the human operator with respect to the eye.

Optionally, the device further comprises:
at least one sensor element operable to measure a distance between the eye and the device,
a control unit coupled to the at least one sensor element, operable to provide a command signal based on the measured distance, to automatically control a relative position between the device and the eye; and
at least an actuator, coupled to the control unit, operable to change the relative position of the device with respect to the eye, based on the command signal received from the control unit.

The term “actuator” as used herein refers to a component that receives a command signal and performs an action based on the received signal in order to change the relative position of the device with respect to the eye. Optionally, this could involve moving the device closer or further away from the eye, or adjusting its angle or orientation. Examples of actuators include motors, solenoids, and piezoelectric devices. In this regard, the device is positioned with respect to the eye by means of the at least one actuator. The command signal applied to the actuator is based on the position feedback obtained by measuring the distance between the device and the eye by means of the at least one sensor element. For example, if the measured distance is more than the desired distance then the control unit is operable to compare the measured distance value with a desired distance value, calculate a position error and deliver a command signal to the actuator based on the position error. The actuator is operable to execute the command by moving the device towards the desired position with respect to the eye. Desired position can be a predetermined distance and direction in respect to eye or direction of pupil of the eye. The predetermined distance depends on a range of action of impact means from the device (i.e for example the range of distances between the device and the cornea that allow to a solid probe to be launched towards the cornea with a predetermined velocity and also that guarantees the re-capture of the solid probe that rebounds after the impact inside the launching means, when a solid probe is used) and also on the operating distance of the measurement unit. Example the predetermined distance can be 5 mm to 20 mm or 5 mm, 10 mm, 15 mm up to 10 mm, 15 mm and 20 mm. Direction can be for example +−10 degrees in respect to normal of surface of the cornea in direction of measurement.

Optionally, the at least one sensor element operable to measure a distance between the eye and the device is selected from any of the following: the confocal chromatic displacement sensor, a laser position sensor and/or an ultrasound position sensor.

A benefit of using the confocal chromatic displacement sensor is that it may be used to perform all the needed measurements: the series of cornea position measurements needed to identify its frequency of oscillation which allows the calculation of the IOP, the cornea thickness and the distance between the device and the cornea needed to correctly position the device relative to the eye.

Alternative or additional position sensors (in addition to CCS) are a laser position sensor, an ultrasound position sensor. One possible advantage of said sensors is low complexity of those (in respect to CCS) thus lower costs. Also for measurement of distance the precision of the measurement does not have to be as accurate as when measuring the oscillations.

Benefit of adding an additional sensor is that if the CCS sensor is designed to meet certain requirements (low cost, high precision, using IR light, small dimensions), then its measuring range of distances may result very small (few mm).

In this case there can be a benefit in adding some other type of additional low-cost sensor such as laser position sensor or ultrasound position sensor. This additional sensor may have a larger range of measured distances with respect to the CCS sensor, therefore it may be useful to help positioning the device relative to the eye when the cornea has not yet entered inside the measuring range of the CCS sensor. The presence of this additional sensor allows the use of a CCS sensor with a reduced measurement range that may be useful to reduce the cost of the CCS sensor or to improve its measuring precision. In this case the additional distance sensor is used to provide a position feedback signal when the cornea is out of the range of measurement of the CCS sensor, for example in the initial phase of the eye examination, when the device is relatively far from its working position relative to the eye.

When the device is finally brought into its working position relative to the eye, the position feedback may continue to be obtained from the measurement of the additional position sensor, or by using the CCS sensor, or both. Therefore, adding a low cost position sensor may allow a large cost reduction of the CCS sensor by reducing its measuring range, but without sacrificing the usability of the device.

According to one embodiment the device is a tonometer. Further optionally the device can be rebound tonometer.

The present disclosure also relates to the method as described above. Various embodiments and variants disclosed above, with respect to the aforementioned first aspect, apply mutatis mutandis to the method.

Optionally, the method further comprises
calculating, using a calculating unit, a frequency of free oscillations of the cornea caused by the impact using the multiple measurements of the displacement of the cornea; and
calculating an internal pressure value of the eye using the frequency of the free oscillations of the cornea caused by the impact.

Optionally, the method further comprises measuring a thickness value of the cornea at least at one point thereon using the measuring unit.

Further optionally, correction to the calculated internal pressure value based on the measured thickness value using the calculating unit might be applied.

Optionally, the method further comprises
measuring a distance between the cornea of the eye and the device, and
using the measured distance as a feedback to place the execution unit at a determined position relative to the cornea. The determined position refers to position which is desirable in respect to obtaining good measurement results. This can be for example 15-20 mm from the eye.

Optionally, the impact on the cornea is created by launching the at least one solid probe towards the eye, the at least one solid probe being comprised in the execution unit.

Optionally, the impact on the cornea is created by launching at least one drop of liquid towards the eye, wherein the at least one drop of liquid is being comprised in the execution unit.

As a summary the device and the method are for measuring properties of eye. The properties can be intra-ocular pressure IOP and/or central corneal thickness (CCT). This measurement is done by applying an impact on the cornea, using impact means, in order to generate a free cornea oscillation after the impact. This oscillation can be measured using measuring unit comprising at least one confocal chromatic displacement sensor. As the measurements of the displacement are done over time (by making multiple samples) it is possible to calculate the frequency of oscillation of the cornea caused by the impact. The said frequency value can be used to calculate intra-ocular pressure. The calculation can be carried out using preset formula or it can be based on lookup table.

### DETAILED DESCRIPTION OF THE DRAWINGS

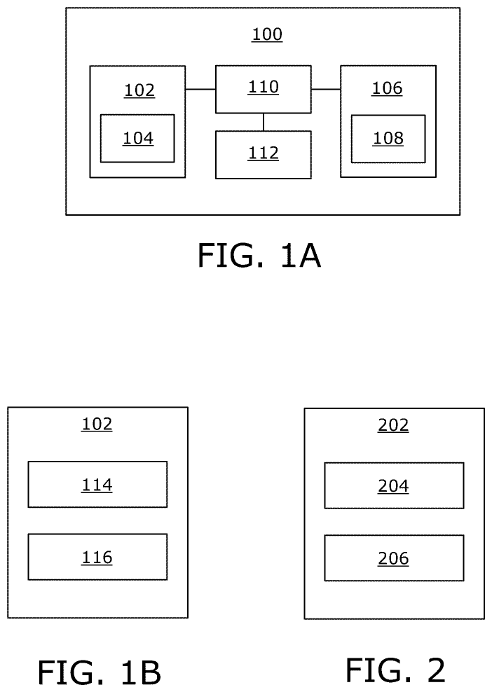

Referring to FIG. 1A, there is shown a block diagram illustrating a device 100 for measuring properties of an eye, in accordance with an embodiment of the present disclosure. As shown, the device 100 comprises an execution unit 102 comprising an impact means 104 arranged to apply an impact on a cornea of the eye. Moreover, the tonometer 100 comprises a measuring unit 106 comprising at least one confocal chromatic displacement sensor 108 and a controller (in some embodiment this controller may be the same one with the “control unit” used to give visual or auditive feedbacks to the operator, or that gives commands to the actuator) 110 coupled to the execution unit 102 and the measuring unit 106. It will be appreciated that the controller 110 is configured to operate the execution unit 102 to apply the impact on the cornea of the eye using the impact means 104 and the measuring unit 106 to perform multiple measurements of a displacement of the cornea caused by the impact during a predetermined time period. Furthermore, the device 100 might comprise a calculating unit 112 coupled to the controller 110. According to one embodiment the calculating unit can be part of the controller. In essence functions such as receiving measurement signals from the sensors, calculating the frequency and the IOP, giving feedback signals to the operator or command actuators, display informations to the operator, save informations etc can be implemented in various configurations, (including remotely from the device) and operable to calculate a frequency of free oscillations of the cornea caused by the impact using the multiple measurements of the displacement of the cornea and calculate an internal pressure value of the eye as a function of the frequency of the free oscillations of the cornea caused by the impact.

Referring to FIG. 1B, there is shown a block diagram illustrating the execution unit 102 of the device 100, in accordance with an embodiment of the present disclosure. As shown, the execution unit 102 further comprises a launching means 114, and at least one solid probe 116 and wherein the launching means 114 is operable to launch the at least the one solid probe 116 towards the eye to create the impact.

FIGS. 1A and 1B are merely examples, which should not unduly limit the scope of the claims herein. A person skilled in the art will recognize many variations, alternatives, and modifications of embodiments of the present disclosure.

Referring to FIG. 2, there is shown a block diagram illustrating an execution unit 202 of a device, in accordance with another embodiment of the present disclosure. As shown, the execution unit 202 further comprises a launching means 204, and at least one drop of liquid 206, and wherein the launching means 204 is operable to launch the at least one drop of liquid 206 towards the eye to create the impact.

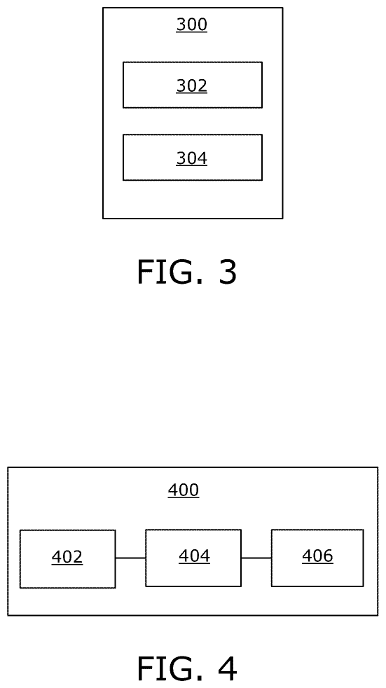

Referring to FIG. 3, there is shown a block diagram illustrating a device 300, in accordance with the other embodiment of the present disclosure. Optionally, the device 300 further comprises at least one sensor element 302 operable to measure a distance between the cornea of the eye and the device 300, and a control unit 304, coupled to the at least one sensor element 302, operable to provide a feedback based on the measured distance. Optionally, the feedback is selected from at least one of: a visual feedback, an acoustic feedback.

Referring to FIG. 4, there is shown a block diagram illustrating a device 400, in accordance with the other embodiment of the present disclosure. Optionally, the device 400 further comprises at least one sensor element 402 operable to measure a distance between the eye (not shown) and the device 400, a control unit 404, coupled to the at least one sensor element 402, operable to provide a command signal based on the measured distance, to automatically control a relative position between the device 400 and the eye. Moreover, the device 400 further comprises at least an actuator 406, coupled to the control unit 404, operable to receive a command signal from the control unit and to change the relative position of the device 400 with respect to the eye.

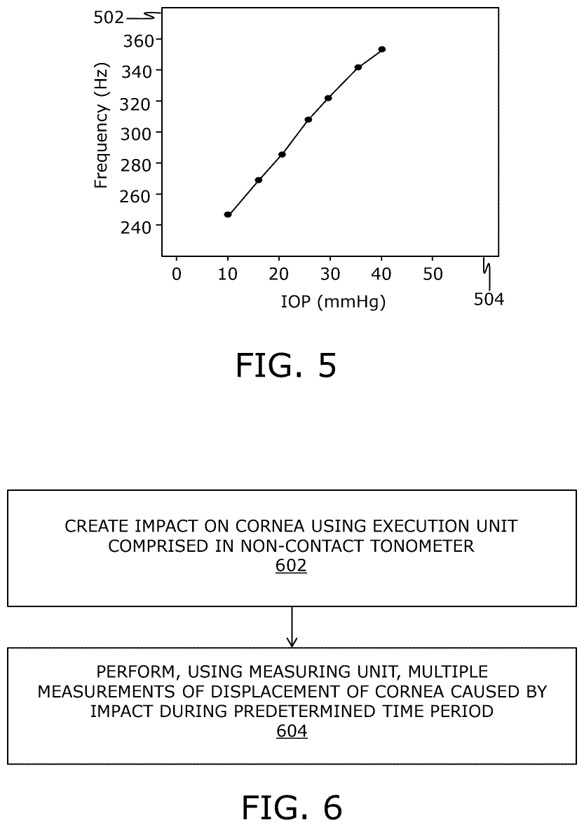

Referring to FIG. 5, there is shown a graphical representation 500 depicting a frequency of the free oscillations of the cornea caused by the impact with respect to the internal pressure value of an eye, in accordance with an embodiment of the present disclosure. As shown, a y-axis 502 depicts the frequency of free oscillations of a cornea caused by an impact, calculated using multiple measurements of a displacement of the cornea. The x-axis 504 depicts an internal pressure value of the eye as a function of the frequency of the free oscillations of the cornea caused by the impact. It will be appreciated that the internal pressure value correlates with the frequency of free oscillations of the cornea.

Referring to FIG. 6, shown is a flowchart depicting steps of a method for measuring properties of an eye, in accordance with an embodiment of the present disclosure. At step 602, an impact on a cornea is created using an execution unit comprised in a device. At step 604, multiple measurements of a displacement of the cornea caused by the impact during a predetermined time period is performed wherein the measuring unit comprising at least one confocal chromatic displacement sensor, wherein the impact on the cornea is created by launching at least one solid probe towards the eye, the at least one solid probe being comprised in the execution unit.

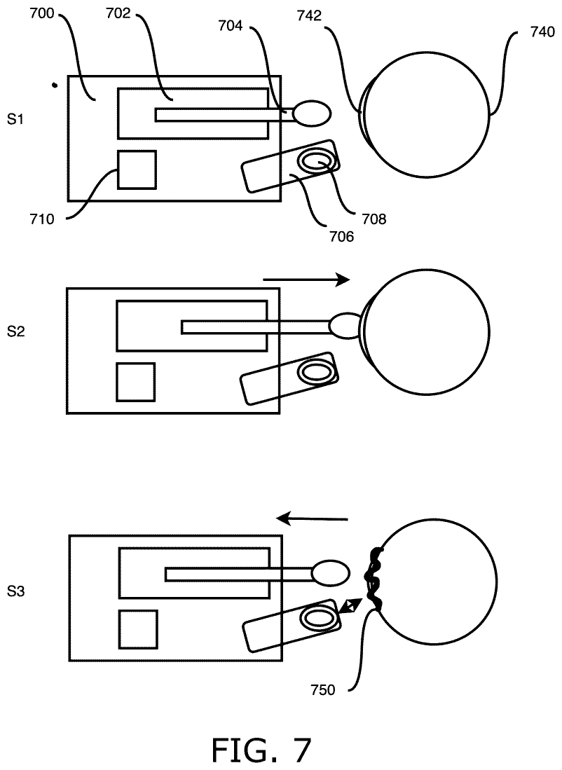

FIG. 7 is an illustration of operation of a device 700 for measuring properties of an eye 740. The device 700 comprises an execution unit 702. The execution unit 702 comprises impact means 704. The impact means 704 in provided example is a probe comprising elongated magnetic body and a tip part as illustrated. The device 700 comprises a measuring unit 706 comprising confocal chromatic displacement sensor 708. A controller 710 is coupled to the execution unit 702 and to the measuring unit 706. As illustrated in the example the execution unit 702 is arranged at least partly inside of body of the device 700. The measuring unit 706 is arranged (connected) on same end of the device body from which the impact means 705 are launched towards eye 740. This way the confocal chromatic displacement sensor 708 can measure the free oscillations of cornea 742 caused by the impact. The confocal chromatic displacement sensor is in other words directed towards the cornea of the eye. Preferably it is directed to the position of impact of the impact means.

In FIG. 7 is illustrated steps S1, S2 and S3 related to operation of the device when used. In S1 the device 700 is placed in desired (determined) distance from cornea 742 of the eye 740 of the patient (user). In step S2 the controller 710 provides control signal for the execution unit 702 to eject impact means 704 (probe in this example) to apply an impact on the cornea 742 of the eye 740. The direction of the movement is indicated with an arrow. The impact means (probe) bounces back (or it is pulled back) as indicated in step S3. The cornea 742 is now oscillating with frequency of free oscillations of the cornea caused on the impact as indicated with oscillations 750. These oscillations correspond to displacement of cornea (surface) caused by the impact. These displacements are measured with the measurement unit 706 during predetermined time period after the impact. This way the frequency can be found out. Since the measuring unit comprises confocal chromatic displacement sensor 708 the corneal thickness can be measured at the same time or after the oscillations have stopped.

The aforementioned steps are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A device for measuring properties of an eye, the device comprising:
an execution unit comprising an impact means arranged to apply an impact on a cornea of the eye,
wherein the execution unit comprises
a launching means, and
at least one solid probe,
a measuring unit comprising at least one confocal chromatic displacement sensor, and
a controller coupled to the execution unit and the measuring unit,
wherein the controller is configured to operate:
the execution unit to apply the impact on the cornea of the eye using the impact means, and
the measuring unit to perform multiple measurements of a displacement of the cornea caused by the impact during a predetermined time period, wherein the launching means is operable to launch the at least one solid probe towards the eye to apply the impact.

2. A device according to claim 1, further comprising a calculating unit coupled to the controller and operable to
calculate a frequency of free oscillations of the cornea, caused by the impact, using the multiple measurements of the displacement of the cornea; and
calculate an internal pressure value of the eye using the frequency of the free oscillations of the cornea caused by the impact.

3. A device according to claim 1, wherein the measuring unit is further configured to measure a thickness value of the cornea at least at one point on the cornea.

4. A device according to claim 2, wherein the calculating unit is further operable to apply a correction to the calculated internal pressure value based on the measured thickness value of the cornea.

5. A device according to claim 1, further comprising
at least one sensor element operable to measure a distance between the cornea of the eye and the device, and
a control unit, coupled to the at least one sensor element, operable to provide a feedback based on the measured distance,
wherein the feedback is selected from at least one of: a visual feedback, an acoustic feedback.

6. A device according to claim 1, further comprising:
at least one sensor element operable to measure a distance between the eye and the device,
a control unit, coupled to the at least one sensor element, operable to provide a command signal based on the measured distance, to automatically control a relative position between the device and the eye; and
at least an actuator, coupled to the control unit, operable to change the relative position of the device with respect to the eye, based on the command signal received from the control unit.

7. A device according to claim 5, wherein the at least one sensor element is selected from any of: the confocal chromatic displacement sensor, a laser position sensor, and/or an ultrasound position sensor.

8. A device according to claim 1, wherein the device is a rebound tonometer.

9. A method for measuring properties of an eye, the method comprising:
creating an impact on a cornea of the eye using an execution unit of a device, and
performing, using a measuring unit of the device, multiple measurements of a displacement of the cornea caused by the impact during a predetermined time period, wherein the measuring unit comprises at least one confocal chromatic displacement sensor,
wherein the impact on the cornea is created by launching at least one solid probe towards the eye, the at least one solid probe being comprised in the execution unit.

10. A method according to claim 9, further comprising
calculating, using a calculating unit, a frequency of free oscillations of the cornea caused by the impact using the multiple measurements of the displacement of the cornea; and
calculating an internal pressure value of the eye using the frequency of the free oscillations of the cornea caused by the impact.

11. A method according to claim 9, further comprising measuring a thickness value of the cornea at least at one point thereon with the measuring unit of the device.

12. A method according to claim 11, further comprising applying a correction to the calculated internal pressure value based on the measured thickness value using the calculation unit.

13. A method according to claim 9, further comprising
measuring a distance between the cornea of the eye and the device, and
using the measured distance as a feedback to place the execution unit of the device at a determined position relative to the cornea.
